(19) United States
(12) Patent Application Publication
(10) Pub. No.: US 2019/0091369 A1
(43) Pub. Date: Mar. 28, 2019

(54) COLLAGEN-BASED DEVICE HAVING ANTIFUNGAL PROPERTIES

(71) Applicant: AROA BIOSURGERY LIMITED, Auckland (NZ)

(72) Inventors: Barnaby Charles Hough MAY, Auckland (NZ); Christopher Hamilton MILLER, Auckland (NZ); Brian Roderick WARD, Waiau Pa (NZ)

(21) Appl. No.: 16/091,761

(22) PCT Filed: Apr. 6, 2017
(86) PCT No.: PCT/NZ2017/050039

§ 371 Date: Oct. 5, 2018

Related U.S. Application Data
(60) Provisional application No. 62/320,761, filed on Apr. 11, 2016.

(51) Int. Cl. A61L 27/24; A61L 27/36; A61L 27/54; A61K 31/65; A61K 35/12; A61K 47/42; A61P 31/10

(52) U.S. Cl. CPC A61L 27/24; A61L 27/3633; A61L 27/54; A61L 2300/406; A61K 35/12; A61K 47/42; A61P 31/10; A61K 31/65

(57) ABSTRACT

A device for wound healing or tissue repair comprising collagen and a tetracycline compound which is effective for preventing or controlling a fungal infection.

### TECHNICAL FIELD

This invention relates to a device useful for promoting the regrowth and healing of damaged or diseased tissue structures. More particularly the invention is directed to a collagen-based device containing a tetracycline where the device exhibits a beneficial antifungal effect.

### BACKGROUND OF THE INVENTION

Collagen-based medical devices have been developed for a wide range of human indications where they serve as structural supports during regeneration of damaged tissue. Collagen-based medical devices additionally provide a temporary matrix that supports the infiltration and attachment of host cells.

Compositions of decellularised tissues from warm-blooded vertebrates, including humans, can be used as tissue graft materials. Common tissue graft compositions may be derived from the dermis, the small intestine, the urinary bladder, renal capsule, the simple glandular stomach and the forestomach matrix (see, for example, U.S. Pat. Nos. 4,902,508, 5,554,389, 6,099,567, 7,087,089, and 8,415,159). These compositions are known as extracellular matrix (ECM) and have an important role in providing the optimal chemical and structural environment for tissue growth and regeneration. ECM scaffolds used for tissue regeneration are traditionally prepared from decellularised human and animal tissues isolated from various organs and from a variety of animal connective tissue or basement membrane sources. These scaffolds promote tissue regeneration and are well-tolerated immunologically.

The inclusion of antimicrobial agents in collagen-based medical devices for the purpose of inhibiting microbial colonisation of the device or to reduce device-related infection is well-known.

The tetracycline antibiotics are a naturally occurring class of antibacterial agents first isolated from Streptomyces species in the late 1940s. Tetracyclines are characterised as exerting antibacterial activity primarily through binding of the bacterial 30S ribosomal subunit causing allosteric inhibition of bacterial peptide synthesis. Tetracycline antibiotics are widely used for the treatment of bacterial infections. Additionally, tetracycline antibiotics such as doxycycline are used for the prophylaxis of plasmodium infections.

Fungal colonisation and infection is an important clinical problem, particularly in patients who are immunocompromised or otherwise at risk of infection. While the antibacterial and antiparasitic properties of doxycycline are well-established, doxycycline is not recognised as having antifungal properties unless present in very high concentrations. For example, doxycycline is active against bacteria at microgram concentrations, but requires milligram concentrations (˜1,000 fold higher concentrations) for activity against fungi. Consequently, doxycycline is not indicated for the treatment of fungal infections.

A study of the antifungal effect of doxycycline against Candida albicans demonstrated that a 5% (50 mg/mL) doxycycline solution was more active than a 17% EDTA solution but less active than a 2.5% NaOCl solution or a 0.2% chlorhexidine gluconate solution.² Doxycycline has a high reported MIC range of 0.64-1.28 mg/mL toward 20 strains of C. albicans.³ High doxycycline concentrations >0.512 mg/mL elicit ≥80% reduction in metabolic activity of C. albicans biofilms. However, this is not correlated with fungicidal efficacy.⁴ Doxycycline has demonstrated “moderate” activity toward inhibiting the germination of fungal spores (50-70% inhibition of fungal spore germination) of the genera Aspergillus, Penicillium and Curvularia.⁵ Doxycycline and tannic acid containing collagen films have demonstrated antimycotic activity against a mixture of yeast and levan genera Candida, Cryptococcus, Histoplasma and Malassezia⁶ where the antifungal activity was attributed to the tannic acid component of the films. In all of these cases, the concentration of doxycycline is several orders of magnitude higher than required for most known antifungal agents. Examples of tetracycline containing medical devices include the XenMatrix™ AB coating which contains both rifampin and the tetracycline antibiotic minocycline for the purpose of preventing bacterial colonisation of the device with no indication of antifungal activity.¹ Thus, doxycycline is not expected to be an effective antifungal agent and would not be selected for this purpose.

Contrary to these expectations, the applicant has found that a collagen-based matrix impregnated with doxycycline shows antifungal activity at clinically relevant concentrations against a range of fungal strains. This development represents the first example of the use of a compound from the class of tetracyclines in a collagen-based device for wound healing and tissue repair.

It is therefore an object of the invention to provide a device for wound healing or tissue repair comprising collagen and a tetracycline compound which overcomes, at least in part, one or more of the abovementioned problems, or to at least provide a useful alternative to existing devices.

### SUMMARY OF THE INVENTION

In a first aspect of the invention there is provided a device for wound healing or tissue repair comprising collagen and a tetracycline compound which is effective for preventing or controlling a fungal infection.

The device may be formed from any suitable collagen containing material, but in preferred embodiments of the invention the device is formed from extracellular matrix (ECM). The ECM may be derived from dermis, pericardium, stomach, small intestine, bladder, placenta, renal capsule, or lining of body cavities of a mammal. In certain embodiments, the ECM is obtained from ovine forestomach. Preferably the ECM is decellularised.

Any tetracycline compound may be used in the device of the invention, such as doxycycline, tetracycline, chlortetracycline, oxytetracycline, demeclocycline, methacycline, minocycline or tigecycline. In some embodiments, the tetracycline compound is doxycycline.

The amount of the tetracycline compound in the device may vary, but typically comprises 0.5% to 10% w/w of the device. In some embodiments, the tetracycline compound comprises 3% to 6% w/w of the device, for example 5% w/w.

The device may be effective for preventing or controlling any fungal infection especially an infection caused by any one or more of Aspergillus niger, Candida albicans, Candida parapsilosis, Candida glabrata and Trichosporon mucoides.

In a second aspect of the invention there is provided the use of a device according to the first aspect of the invention for wound healing or tissue repair. In some embodiments of the invention, the device is surgically fixed to animal tissue or implanted into animal tissue.

### BRIEF DESCRIPTION OF THE FIGURE

FIG. 1 shows the antifungal activity of a collagen-based device material containing 5% doxycycline against fungal pathogens.

### DETAILED DESCRIPTION

The term “extracellular matrix” (ECM) as used herein refers to animal or human tissue that has been decellularised and provides a matrix for structural integrity and a framework for carrying other materials.

The term “decellularised” as used herein refers to the removal of cells and their related debris from a portion of a tissue or organ, for example, from ECM.

The term “collagen” as used herein refers to the main structural protein in the extracellular space in various connective tissues in animal bodies. As the main component of connective tissue, it is the most abundant protein in mammals making up from 25% to 35% of the whole-body protein content.

The term “tetracycline” as used herein refers to a group of broad-spectrum antibiotics defined as “a subclass of polyketides having an octahydrotetracene-2-carboxamide skeleton”. They are collectively known as “derivatives of polycyclic naphthacene carboxamide”. They include doxycycline, tetracycline, chlortetracycline, oxytetracycline, demeclocycline, methacycline and others. Tetracyclines remain the treatment of choice for infections caused by chlamydia (trachoma, psittacosis, salpingitis, urethritis and L. venerum infection), Rickettsia (typhus, Rocky Mountain spotted fever), brucellosis and spirochetal infections (borreliosis, syphilis and Lyme disease). In addition, they may be used to treat anthrax, plague, tularemia and Legionnaires' disease. They are also used in veterinary medicine.

Tetracyclines have the following general core chemical structure:

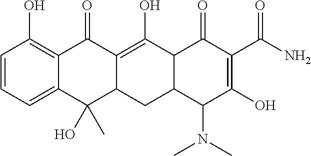 [SMILES: CN(C)C1C(O)=C(C(N)=O)C(=O)C2C(O)=C3C(=O)c4c(O)cccc4C(C)(O)C3CC21]

Doxycycline is an antibiotic that is used in the treatment of a number of types of infections caused by bacteria and protozoa. It is not a known antifungal agent. Doxycycline has the following chemical structure:

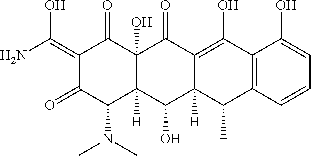 [SMILES: C[C@H]1c2cccc(O)c2C(O)=C2C(=O)[C@]3(O)C(=O)/C(=C(/N)O)C(=O)[C@@H](N(C)C)[C@@H]3[C@@H](O)[C@@H]21]

The invention broadly relates to a device for wound healing or tissue repair comprising collagen and a tetracycline compound which is effective for preventing or controlling a fungal infection.

The applicant has found that a collagen-based matrix impregnated with the tetracycline compound doxycycline exhibits antifungal activity across a broad range of fungal strains. It is expected that other compounds from the same class of tetracyclines when incorporated into a collagen-based matrix such as ECM will also show antifungal activity. The invention therefore relates to any collagen-based medical device in combination with any tetracycline.

In preferred embodiments of the invention, the device is formed from extracellular matrix (ECM). ECM may be obtained from any suitable source, for example sheep forestomach. Typically, the ECM will be decellularised so that the risk of any immune response when used in an animal body is avoided or minimised.

ECM-derived matrices for use in the invention are collagen-based biodegradable matrices comprising highly conserved collagens, glycoproteins, proteoglycans and glycosaminoglycans in their natural configuration and natural concentration. One extracellular collagenous matrix for use in this invention is ECM of a warm-blooded vertebrate. ECM can be obtained from various sources, for example, intestinal tissue harvested from animals raised for meat production, including pigs, cattle and sheep or other warm blooded vertebrates. Vertebrate ECM is a plentiful by-product of commercial meat production operations and is thus a low cost tissue graft material.

The ECM tissue suitable for use in the formation of the graft products comprises naturally associated ECM proteins, glycoproteins and other factors that are found naturally within the ECM depending upon the source of the ECM.

Forestomach tissue is a preferred source of ECM tissue for use in this invention. Suitable forestomach ECM typically comprises the propria-submucosa of the forestomach of a ruminant. In particular embodiments of the invention, the propria-submucosa is from the rumen, the reticulum or the omasum of the forestomach. These tissue scaffolds typically have a contoured luminal surface. The ECM tissue scaffold may additionally contain decellularised tissue, including portions of the epithelium, basement membrane or tunica muscularis, and combinations thereof. The tissue scaffolds may also comprise one or more fibrillar proteins, including but not limited to collagen I, collagen III or elastin, and combinations thereof.

Propria-submucosa tissue typically has an abluminal and a luminal surface. The luminal surface is the surface facing the lumen of the organ source and the abluminal surface faces the smooth muscle tissue surface. Multiple sheets of propria-submucosa can be overlapped with the abluminal surface contacting the luminal surface, the luminal surface contacting the luminal surface, or with the abluminal surface contacting the abluminal surface of an adjacent sheet of ECM. All of these combinations of overlapping sheets of ECM from some or different vertebrate or organ sources will produce a laminated graft product comprising ECM.

One method of preparing ECM for use in accordance with this invention is described in U.S. Pat. No. 8,415,159. A segment of the vertebrate forestomach, preferably harvested from ovine species is subjected to a transmural osmotic flow between two sides of the tissue, such that the tissue layers within all or a portion of the tissue are separated and/or decellularised. The transmural osmotic flow can be directed from the luminal to the abluminal side of all or a portion of the tissue, or from the abluminal to the luminal side of all or a portion of the tissue. This may be achieved, for example, by separating the tissue between a hypertonic and a hypotonic solution, such that the transmural osmotic flow is directed from the hypotonic solution to the hypertonic solution.

The method may further involve removing all or part of a tissue layer including epithelium, basement membrane, or tunica muscularis, and combinations thereof. The hypertonic and hypotonic solutions may include, for example, water and optionally at least one buffer, detergent or salt. The hypertonic solution contains a higher concentration of solute than the hypotonic solution. In a particular method, the hypertonic solution comprises 4 M NaCl and the hypotonic solution comprises 0.28% Triton X-200 and 0.1% EDTA. In another particular method, the hypotonic solution comprises 0.1% SDS. In still another method, the hypotonic solution comprises 0.028% Triton X-200, 0.1% EDTA, and 0.1% SDS. The ECM can be stored in a hydrated or dehydrated state. Lyophilised or air dried ECM may be rehydrated or partially rehydrated and used in accordance with this invention without significant loss of its biotropic and mechanical properties.

Although any tetracycline may be used in the device of the invention, the preferred tetracycline is doxycycline. Others include, but are not limited to, tetracycline, chlortetracycline, oxytetracycline, demeclocycline, methacycline, minocycline and tigecycline.

The tetracycline may be present in any suitable amount to give a desired antifungal effect. In a typical device of the invention, the tetracycline comprises 0.5% to 10% w/w of the device, preferably 3% to 6% w/w, e.g. 5% w/w.

Although tested against the five fungi Aspergillus niger, Candida albicans, Candida parapsilosis, Candida glabrata and Trichosporon mucoides, it will be appreciated that the device of the invention may be effective against any fungal infection.

Example 1 describes the preparation of a doxycycline containing collagen-based medical device. Example 2 describes the assessment of the device material for antimicrobial effectiveness against five species of fungi which are clinically relevant to the colonisation and infection of wounds. The doxycycline containing material exhibited an antimicrobial effectiveness of >5 log reduction against C. albicans, C. glabrata and T. mucoides, and an antimicrobial effectiveness of ˜3 log reduction against C. parapsilosis and A. niger. Both of these log reduction values indicate a clinically useful antifungal effectiveness in preventing the colonisation of the device material or preventing device related infection. Although antibacterial effectiveness of the material would be expected because of the known antibacterial properties of doxycycline, the effectiveness of the material against fungi was unexpected. Accordingly, the applicant's finding represents the first use of a tetracycline incorporated into a medical device used for tissue repair which is clinically useful in the prevention and/or treatment of fungal infections.

It will be appreciated that in many instances of infection at the site of wound healing or tissue repair it is not known whether the infection is a bacterial infection or a fungal infection (or any other type of infection, such as a viral infection). In such infections of unknown etiology, the device of the infection will be clinically relevant because whether or not a bacterial infection is present there may also be a co-existing fungal infection or at least the need to prevent a co-existing fungal infection from occurring. Thus, the device of the invention is useful for treating a microbial infection provided the microbial infection is or includes a fungal infection or at least a clinician determines that there is a need to prevent a fungal infection (whether or not in addition to any other type of microbial infection).

Any reference to prior art documents in this specification is not to be considered an admission that such prior art is widely known or forms part of the common general knowledge in the field.

As used in this specification, the words “comprises”, “comprising”, and similar words, are not to be interpreted in an exclusive or exhaustive sense. In other words, they are intended to mean “including, but not limited to.

The invention is further described with reference to the following examples. It will be appreciated that the invention as claimed is not intended to be limited in any way by these examples.

### EXAMPLES

### Example 1: Preparation of Doxycycline Containing Collagen-Based Device Material

ECM from sheep forestomach was processed to decellularise the tissue in accordance with the procedure described in U.S. Pat. No. 8,415,159. Doxycycline was incorporated at a target concentration of 5% w/w in the device material by performing a buffer exchange on the ECM material to replace residual buffer with an appropriate buffer for solubilisation of doxycycline. ECM tissue was added to the buffer exchange solution and mixed for 10 minutes. After draining excess liquid from the ECM tissue, the tissue was soaked in an aqueous doxycycline solution and mixed until saturation of the tissue with doxycycline. The tissue was drained of excess doxycycline solution and lyophilized to produce dry material with a doxycycline concentration of 5% w/w. Forestomach tissue without doxycycline was also lyophilised in order to compare the effect of doxycycline on the biophysical performance of the ECM.

### Example 2: Antifungal Effect of Doxycycline Containing Collagen-Based Device Material

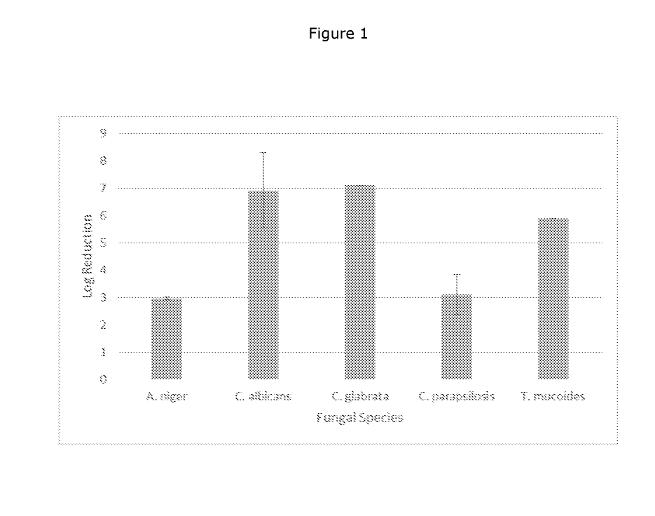

The doxycycline containing ECM tissue prepared in accordance with Example 1 and lyophilised ECM tissue containing no doxycycline were assessed in triplicate for antifungal activity against the clinically relevant fungal species Aspergillus niger, Candida albicans, Candida parapsilosis, Candida glabrata and Trichosporon mucoides using a 24 hour contact period. The procedure followed is described in “15020743 Textiles—Determination of antibacterial activity of antibacterial finished products (absorption method).” The results were recorded as an average log reduction between the doxycycline treated material (n=3) and the non-doxycycline treated control (n=3) and are shown in FIG. 1. The results demonstrate that the collagen-ECM medical device material containing doxycycline exhibits potent and unexpected antifungal activity.

Although the invention has been described by way of example, it should be appreciated that variations and modifications may be made without departing from the scope of the invention as defined in the claims. Furthermore, where known equivalents exist to specific features, such equivalents are incorporated as if specifically referred in this specification.

### REFERENCES

1. http://www.davol.com/sp/xenmatrix-ab-surgical-graft/2.
2. Lau, H., et al. (2008). “Evaluation of antifungal efficacy of 5% doxycyline hydrochloride, 2.5% sodium hypochlorite, 17% ethylenediamine tetraacetic acid and 0.2% chlorhexidine gluconate against Candida albicans.” An in vitro study. Endotontology 20: 6-13.
3. Lew, M. A., et al. (1977). “Antifungal activity of four tetracycline analogues against Candida albicans in vitro: potentiation by amphotericin B.” Journal of Infectious Diseases 136(2): 263-270.
4. Miceli, M. H., et al. (2009). “In vitro analyses of the combination of high-dose doxycycline and antifungal agents against Candida albicans biofilms.” International Journal of Antimicrobial Agents 34(4): 326-332.
5. Prasad, S. and H. Nema (1982). “Mycotic infections of cornea.” Indian Journal of Ophthalmology 30(2): 81.
6. Albu, M., et al. (2010). “Doxycycline delivery from collagen matrices crosslinked with tannic acid.” Molecular Crystals and Liquid Crystals 523(1): 97/[669]-105/[677].

1. A device for wound healing or tissue repair comprising collagen and a tetracycline compound which is effective for preventing or controlling a fungal infection.

2. A device as claimed in claim 1, which is formed from extracellular matrix (ECM).

3. A device as claimed in claim 2, wherein the ECM is derived from dermis, pericardium, stomach, small intestine, bladder, placenta, renal capsule, or lining of body cavities of a mammal.

4. A device as claimed in claim 2, wherein the ECM is obtained from ovine forestomach.

5. A device as claimed in claim 2, wherein the ECM is decellularised.

6. A device as claimed in claim 1, wherein the tetracycline compound is doxycycline, tetracycline, chlortetracycline, oxytetracycline, demeclocycline, methacycline, minocycline or tigecycline.

7. A device as claimed in claim 1, wherein the tetracycline compound is doxycycline.

8. A device as claimed in claim 1, wherein the tetracycline compound comprises 0.5% to 10% w/w of the device.

9. A device as claimed in claim 8, wherein the tetracycline compound comprises 3% to 6% w/w of the device.

10. A device as claimed in claim 1, which is effective for preventing or controlling a fungal infection caused by any one or more of Aspergillus niger, Candida albicans, Candida parapsilosis, Candida glabrata and Trichosporon mucoides.

11. The use of a device according to claim 1 for wound healing or tissue repair.

12. The use as claimed in claim 11, wherein the device is surgically fixed to animal tissue or implanted into animal tissue.

13. The use as claimed in claim 11, for the treatment or prevention of a fungal infection.

14. A device for wound healing or tissue repair comprising collagen and a tetracycline compound which is effective for preventing or controlling a microbial infection provided the microbial infection is or includes a fungal infection or is likely to include a fungal infection.

15. The use of a device as claimed in claim 14 for preventing or controlling a microbial infection provided the microbial infection is or includes a fungal infection or is likely to include a fungal infection.
